(19) United States
(12) Patent Application Publication
(10) Pub. No.: US 2024/0288418 A1
(43) Pub. Date: Aug. 29, 2024

(54) APPARATUS FOR ANALYSING A BIOLOGICAL SAMPLE FOR DIAGNOSTICS PURPOSES

(71) Applicant: MICRONIT HOLDING B.V., ENSCHEDE (NL)

(72) Inventor: Ronny VAN 'T OEVER, ENSCHEDE (NL)

(73) Assignee: MICRONIT HOLDING B.V., ENSCHEDE (NL)

(21) Appl. No.: 18/573,585

(22) PCT Filed: Jul. 11, 2022
(86) PCT No.: PCT/NL2022/050401

§ 371 Date: Dec. 22, 2023

(30) Foreign Application Priority Data
Jul. 14, 2021 (NL) 2028727

(51) Int. Cl. G01N 33/52; B01L 3/00; B01L 7/00

(52) U.S. Cl. CPC G01N 33/528; B01L 3/502715; B01L 7/52; B01L 2200/04; B01L 2300/023; B01L 2300/0663; B01L 2300/168; B01L 2300/18

(57) ABSTRACT

An apparatus for analysing a biological sample for diagnostics purposes includes a sample cartridge receptacle for receiving a sample cartridge for holding the biological sample. The sample cartridge includes at least two sample reaction zones for containing the biological sample. At least one light source for illuminating the at least two sample reaction zones of a sample cartridge when received in the sample cartridge receptacle. At least two measurement sections each arranged to perform measurements on a respective sample reaction zone when received in the sample cartridge receptacle, where each measurement section includes a light detector for receiving light illuminated onto the respective sample reaction zone by the at least one light source. A processing unit for controlling the at least one light source and for receiving data from the measurement sections.

The present invention relates to an apparatus for analysing a biological sample for diagnostics purposes, a system for analysing a biological sample for diagnostics purposes comprising such an apparatus and a sample cartridge for use in an apparatus. The invention further relates to a method for analysing a biological sample.

Apparatuses or devices which allow consumers to perform tests on biological samples such as saliva, blood or the like are known. It is for instance known to apply the sample on a sample cartridge which is then inserted into the apparatus which is provided with a measurement section for performing measurements on the sample. Such a device may then be provided with a processing unit coupled to the measurement section to process the data received from the measurement section to analyse the sample, for instance for diagnostic purposes.

It is a drawback of these known apparatuses that they are typically limited to one type of biological sample and (diagnostic) test. Moreover, for some of the tests, the reliability, for instance in terms of sensitivity and/or selectivity, of the outcome may be improved.

It is a goal of the present invention, next to other goals, to provide an improved apparatus for analysing a biological sample, wherein at least one of above mentioned drawbacks is at least partly alleviated.

This goal, amongst other goals, is met by a method according to appended claim 1. More specifically, this goal, amongst other goals, is met by an apparatus for analysing a biological sample for diagnostics purposes, wherein the apparatus comprises:
a sample cartridge receptacle for receiving a sample cartridge for holding the biological sample, wherein the sample cartridge comprises at least two sample reaction zones for containing said biological sample;
at least one light source associated with the sample cartridge receptacle for illuminating the at least two sample reaction zones of a sample cartridge when received in the sample cartridge receptacle;
at least two measurement sections associated with the sample cartridge receptacle, wherein each of the at least two measurement sections is arranged to perform measurements on a respective sample reaction zone of the at least two sample reaction zones of a sample cartridge when received in the sample cartridge receptacle, wherein each measurement section comprises at least one light detector for receiving light illuminated onto the respective sample reaction zone by the at least one light source;
a processing unit arranged for controlling the at least one light source and for receiving data from the measurement sections.

By providing at least two measurement sections, associated with the two reaction zones in the cartridge in inserted position, multiple measurements can be performed on the sample cartridge, even at the same time. The measurement sections and the processing unit are preferably arranged to individually control the measurement sections for performing independent measurements on the reaction zones.

The sample cartridge may be arranged to contain a single biological sample, wherein the reaction zones contain the same biological sample. As such, multiple measurements can be performed on the same sample in a single apparatus in separate sample reaction zones. A versatile and reliable apparatus for analysing samples is thus provided. It may also be possible that at least two of the measurement sections perform the same measurement, although on different reaction zones. The reliability of the apparatus may thus be improved. A combination of different measurements and redundant measurements may be employed.

It is noted that although the apparatus is particularly suitable to analyse biological samples and/or specimens, preferably for diagnostic or generally monitoring purposes, the apparatus and corresponding sample cartridge may also be employed to analyse other types of samples. Further, although the cartridge preferably contains sample reaction zones, in which zones a chemical reaction with the sample may occur to detect the presence of and/or quantify a molecule or other substance of interest, the cartridge or apparatus may also generally contain a sample holder for holding a sample. A measurement section may then be arranged to perform measurements on the sample held in the sample holder.

The cartridge may for instance comprise at least one reaction chamber for containing a sample and possibly a reagent as reaction zone. Preferably a plurality of reaction chambers is provided. At least two reaction chambers may then contain two different reagents or are more generally arranged to perform two different tests alternatively or additionally, at least two reaction chambers may contain the same reagent or may be generally arranged to perform two identical tests.

Additionally or alternatively, the sample cartridge may comprise at least one lateral flow assay configuration as reaction zone. A measurement section may then be arranged to perform measurements on at least one test zone, for instance in the form of a test line, dot or other pattern arranged to change colour in dependence of the presence of a molecule of interest, of such a lateral flow assay configuration. As such, a test zone of the lateral flow assay may form a reaction zone onto which measurements may be performed with a measurement section. The lateral flow assay configuration may comprise a plurality of test zones, for instance at least one test line and a control line. A single lateral flow assay configuration may thus form a plurality of reaction zones in the form of the plurality of test zones. The apparatus may then be arranged to perform measurements on the respective test zones or reaction zones of the lateral flow assay configuration.

To improve the reliability and versatility of the measurements by the light detector, it is preferred if the reaction zones are illuminated. A light source is thus preferably provided which is arranged for illuminating at least one sample reaction zone of a sample cartridge when received in the sample cartridge receptacle. Preferably a plurality of reaction zones is illuminated.

Although a single light source can be used for illuminating more than one reaction zone, preferably a plurality of light sources is provided. At least two of the measurement sections, preferably each measurement section, comprise a respective light source for illuminating the respective reaction zone of a sample cartridge when received in the sample cartridge receptacle. This allows adjusting the light to the measurement to take place in the reaction zone.

To allow performing a plurality of tests, the apparatus is preferably arranged to illuminate light at a reaction zone, preferably a plurality thereof, having different wavelengths. On the basis of the test to be performed, light with a predetermined wavelength can then be illuminated on the reaction zone, which can then be efficiently measured by a light detector in a measurement section. The wavelength of the light irradiated onto the reaction zone need not to correspond to the wavelength of the detected light.

It may be possible that one light source is arranged to emit light having different wavelengths. A preferred embodiment of the apparatus however comprises a plurality of light sources, wherein at least two of the light sources are arranged to emit light with different wavelengths. Relative cheap light sources, for instance LEDs, can then be used, which are also energy efficient.

Preferably, the apparatus comprises at least one light source, for instance in a measurement section, for emitting red light, for instance light having a maximum intensity at a wavelength around 610 nm and/or 620 nm, green light, for instance light having a maximum intensity at a wavelength around 520 and/or 525 nm, and/or blue light, for instance light having a maximum intensity at a wavelength around 450 and/or 460 nm. One light source may be arranged to emit light having a specific colour (range), i.e. a monochromatic light. A cheap configuration is thus obtained if single colour light sources are used, for instance in the form of LEDs. A RGB light source may also be provided which is arranged to emit light having a plurality of different wavelengths.

Preferably, also a light source arranged to emit generally white light is provided, for instance having a colour temperature around 5700K. A monochromatic light source may hereto be provided. Additionally or alternatively, a RGB light source may be arranged to emit substantially white light. It is also possible to provide a RGBW light source, for instance in the form of a RGBW LED, which is arranged to emit both red, green and blue and white light. By providing light sources for emitting substantially white light, the apparatus is particularly suitable to perform measurement on a sample cartridge having a lateral flow assay configuration, or to perform measurements on lateral flow assay configurations generally.

Preferably, the apparatus comprises both monochromatic light sources, for instance in the form of LEDs, and at least one RGB light source arranged to emit light having a plurality of different wavelengths. A relatively cheap yet versatile apparatus is thus provided. More preferably, the apparatus comprises a plurality of monochromatic light sources, for instance in the form of a plurality of LEDs, for emitting light with a first set of wavelengths, for instance 610 nm or 620 nm, 520 or 525 nm and 450 or 460 nm, wherein the RGB light source is arranged to emit the other of the 610 nm or 620 nm, 520 or 525 nm and 450 or 460 nm.

Preferably, the apparatus is further provided with a light source arranged to emit UV light. Providing such a UV-light source, for instance arranged to emit light having a (maximum intensity at a) wavelength of approximately 367 nm, improves the versatility of the apparatus.

According to a further preferred embodiment, at least two measurement sections comprise at least two light sources arranged to emit light with different wavelengths. The two measurement sections can then each illuminate light with different wavelengths, thereby improving the versatility of the apparatus.

According to a further preferred embodiment, the processing unit is arranged to individually control the at least two light sources for illuminating the at least two sample reaction zones, preferably with light having different wavelengths. The wavelength can then be adapted to the test performed in a respective reaction zone. Preferably, the processing unit is further arranged to receive data from the respective measurement sections obtained from illumination of the respective sample reaction zones illuminated with light having mutually different wavelengths. The output of the different measurement sections is fed to the processing unit, where the data can be processed and/or output.

According to a further preferred embodiment, the apparatus is arranged to simultaneously perform measurements on two sample reactions zones of a sample cartridge when received in the sample cartridge receptacle. As mentioned, this allows performing measurement on a sample, for instance the same sample, simultaneously, instead for instance sequentially, which saves time and/or allows more accurate tests. For instance when the cartridge comprises two reaction zones for performing the same test on the same sample, performing measurements on both reaction zones improves the precision. Performing measurements on two different reaction zones, i.e. arranged to perform different tests, improves the versatility and (time) efficiency. Preferably, the processing unit is hereto arranged to control at least two measurement sections for simultaneously taking measurements on two sample reactions zones of a sample cartridge when received in the sample cartridge receptacle.

Efficient and versatile measurements can be performed when the light detector comprises a multi-spectral sensor. Light of different wavelengths can then be measured, such that different tests can be performed in a single measurement section. It may for instance be the case that a single reaction zone comprises at least two different test or assays, which each require measurement of light having a different wavelength, for instance by detecting fluorescence and general colorimetric detection. The multi-spectral sensor is then arranged to detect the assay results, e.g. the emission of light of at least two different wavelengths, of both tests in said reaction zone.

Preferably, the light detector comprises a multi-channel spectral colour sensor, preferably arranged to detect light in at least the visible spectrum, preferably also in the UV and/or IR spectrum. The processing unit is then preferably arranged to receive multi-spectral data from the sensor.

Two measurement sections may have substantially the same configuration. The apparatus can then be manufactured easily, while each of the sections offers a wide range of tests.

According to a preferred embodiment, at least two of the measurement sections have a different configuration. This allows customizing the measurement sections to specific tests, while using relatively cheap parts. Two different measurement sections may for instance comprise light sources for emitting light with different wavelengths. A first measurement section may for instance comprise a light source for emitting light having a first wavelength, while a second measurement section may comprise a light source for emitting light having a second wavelength, different from said first light source. Said second measurement section preferably then not comprises a light source emitting light having the first wavelength.

As mentioned, providing a plurality of measurement sections allows performing a plurality of tests simultaneously. It is however noted that it is also envisaged to provide an apparatus having only a single measurement section, for instance having the plurality of light sources as mentioned above and/or the multi-spectral sensor. Also such an apparatus still allows multiple tests. As mentioned above, it may for instance be possible that in one reaction zone multiple excitations occur. The light detector, preferably the multi-spectral sensor as mentioned above, may then be arranged to detect light having different wavelengths corresponding to the different excitation reactions. A reaction zone may then also be illuminated with light having different wavelengths. A plurality of light sources may thereto be provided and/or a RGB light source arranged to emit light with a predetermined or adjustable colour as mentioned above.

A further preferred embodiment of the apparatus further comprises a frame member, wherein the light source and the light detector are arranged on the frame member. Preferably, the frame member comprises a Printed Circuit Board (PCB). This reduces the number of parts and thus allows an efficient configuration.

According to a preferred embodiment, the frame member, preferably in the form of a PCB, comprises a plurality of light detectors and light sources for defining a plurality of measurement sections. Preferably, the sample cartridge receptacle is at least partly defined by the frame member, for instance in the form of a PCB.

According to a further preferred embodiment, the apparatus further comprises a light shielding mechanism arranged between at least two components of a measurement section or between measurement sections. Interference of components is hereby reduced. The light shielding mechanism may comprise a light shielding wall extending between two components, for instance between at least one light source and a light sensor, preferably in a measurement section. This increases the signal-to-noise ratio. More preferably, a light shielding mechanism, for instance in the form of a light shielding wall, is provided between measurement sections to reduce any mutual influences of the measurement sections, in particular any light sources thereof.

To prevent any contact between the reaction zones, and in particular any sample present therein, and the electronic components, it is preferred if the sample cartridge receptacle is at least partly defined by a transparent member, preferably a plate shaped member, arranged between at least the light detector in a measurement section and a sample cartridge when received in the sample cartridge receptacle. Preferably, the transparent member comprises a heat reflecting layer. This protects the components from any heat, as will be discussed also in greater detail below.

A further preferred embodiment of the apparatus further comprises at least one heating element arranged for heating a respective sample reaction zone of a sample cartridge when received in the sample cartridge receptacle, wherein the processing unit is preferably arranged for controlling the at least one heating element. This allows heating the reaction zone, which for instance allows thermal cycling in a PCR-test. Preferably, the heating element is arranged to heat the reaction zone to a plurality of different temperatures. It will be appreciated that the heating element as described may also be employed in an apparatus having only one measurement section as described earlier.

One heating element may be arranged to heat a plurality of reaction zones. However, to be able to accurately control the temperature in a reaction zone, preferably at least two measurement sections have associated therewith a respective heating element arranged for heating the respective sample reaction zones of a sample cartridge when received in the sample cartridge receptacle. The processing unit is then preferably arranged for individually controlling the at least two heating elements. Different heating elements may for instance heat the respective reaction zones to different temperatures. The heating elements are preferably aligned with the reaction zones of a sample cartridge, when received in the receptacle.

To improve the heating of a sample in a reaction zone, which may be shaped as a sample chamber, a reaction zone preferably comprises a heat conducting section for cooperating with a heating element. In inserted position, the heat conducting section of the reaction zone is then aligned with a heating element. More preferably, a reaction zone comprises a substantially planar wall of heat conducting material.

A reaction zone may for instance comprise a reaction chamber. At least one of the walls of the chamber may then be formed to improve the heat conduction between the sample and the heating element in inserted position of the sample cartridge. More preferably, the reaction chamber comprises a first wall and a second wall, preferably interconnected by at least one circumferential wall for providing a substantially enclosed reaction chamber. At least one of the walls may be arranged to allow measurement therethrough. This wall may for instance be formed at least partially transparent. In inserted position, this wall is then preferably aligned with a light detector of a measurement section. Further, one of the walls, preferably the wall opposite the wall for allowing measurement, is formed as a heat conducting section and is thereto preferably shaped substantially planar.

To reduce any influence, in terms of heating, of the heated reaction zones on in particular the light detector, a further preferred embodiment of the apparatus further comprises a heat sink associated with the light detector. This reduces any sensor drift.

The receptacle is preferably slot shaped and is shaped to closely receive the cartridge. The sample cartridge and the receptacle are therefore preferably formed as least partly complementary. Preferably, the receptacle is shaped such that the cartridge only fits the receptacle in one way. In other words, when correctly, for instance fully inserted, the relative position of the reaction zones in the apparatus is fixed. This allows efficient aligning of the measurement sections and possibly the heating elements with the reaction zones when the cartridge is received in the receptacle.

Preferably, the measurement section is arranged at first side of the sample cartridge receptacle and wherein the heating element, or the plurality thereof, is arranged at an opposite side of the sample cartridge receptacle for receiving a sample cartridge therebetween. Measurement can then efficiently take place from one side, while the other side can be heated.

According to a further preferred embodiment, the measurement section is arranged on a first frame member, for instance in the form of a PCB, and wherein the heating element is arranged on a second frame member, for instance in the form of a PCB, wherein the sample cartridge receptacle is defined between the first and second PCBs.

According to a further preferred embodiment, the sample cartridge receptacle is arranged for receiving a sample cartridge comprising a plurality of sample reaction zones for containing said biological sample, wherein the reaction zones are arranged in a two-dimensional array configuration. A plurality of reaction zones can then be provided, while still providing a relatively compact cartridge and therewith a more compact apparatus. Preferably, the reaction zones are separated in a first direction and a second direction different, preferably orthogonal, to the first direction. The reactions are then preferably arranged in an array, more preferably at mutual distances.

A further preferred embodiment comprises a plurality of measurement sections, wherein the measurement sections are arranged in a corresponding two-dimensional array configuration. When the apparatus is provided with heating elements as described above, the heating elements are preferably also arranged in a corresponding two-dimensional array configuration. It is also possible that the reaction zones, and thus preferably the measurement sections and any heating elements are arranged in different two-dimensional configurations, such as a hexagonal configuration.

A further preferred embodiment further comprises a memory, wherein the memory at least temporally, preferably at least prior to measuring, contains instructions for controlling the light source, the detection section and optionally the heating element, for performing predetermined measurements on a reaction zone of a received sample cartridge. In dependence of the test to be performed on a particular reaction zone, the light source, the light detector and possibly the heating element may then be controlled in accordance with the test to be performed. As mentioned, different tests may be performed simultaneously at different measurement sections, such that each of these measurement sections may then receive their respective instructions.

Preferably, the apparatus is arranged to perform a plurality of different measurements including at least one, preferably at least two, of a PCR test, LAMP test, antigen test, ELISA, pH test, a (neutralizing) antibody test and any other reaction based tests such as based on isothermal (amplification) reactions, chemical reaction, redox reactions or generally any reaction that results in a change in the optical properties of a sample. A memory may for instance contain and/or receive instructions for performing the above mentioned tests.

A further preferred embodiment further comprises an output module for outputting the received data from the measurement sections. Preferably, the output module is a wireless module for wirelessly transmitting the data, for instance based on WiFi (WLan), Bluetooth or the like. Preferably, all data, for instance in aggregated form, received by the light detectors is output. The output module may be arranged to output the received multi-spectral data in a multiplexed manner.

According to a preferred embodiment, the apparatus is a handheld device, preferably further comprising a battery, preferably a rechargeable battery.

According to a further aspect, a system for analysing a biological sample for diagnostics purposes is provided, wherein the system comprises an apparatus as described above and a central server, wherein the apparatus is arranged to provide the received data from the measurement sections to the central server, wherein the central server is arranged to analyse the received data and to provide a diagnostic parameter being indicative for the results of the diagnostic test based on the sample in the sample cartridge. By determining the parameter centrally, less computing power is required in the apparatus or possibly an electronical device such as a smartphone, laptop or the like, of the user. Preferably, the central server comprises an output for outputting the diagnostic parameter to a user. The parameter may be output via a webserver or other internet connected manner.

More preferably, the central server is arranged to receive the data via an electronical device from a user, such as a mobile phone, a tablet or a laptop, coupled to the apparatus. The apparatus may for instance be coupled to the electronical device, for instance via Bluetooth, wherein the electronical device transmits the data to the server. Most preferably, the output is arranged to provide the parameter to the electronical device from the user.

According to a further aspect, a sample cartridge is provided, in particular for use in an apparatus as described above, wherein the sample cartridge comprises at least two sample reaction zones for containing said biological sample. Preferably, the reaction zones are arranged in a two-dimensional array configuration, wherein the reaction zones are separated in a first direction and a second direction, preferably orthogonal to the first direction.

According to a preferred embodiment, at least two reaction zones are arranged for two different assays or tests. Preferably, at least one of the reactions zones is arranged for a PCR test, LAMP test, antigen test, ELISA, pH test, a (neutralizing) antibody test and any other reaction based tests such as based on isothermal (amplification) reactions, chemical reaction, redox reactions or generally any reaction that results in a change in the optical properties of a sample.

It should however be mentioned that although the apparatus as described above works particularly well in combination with a sample cartridge, it may also be possible that the apparatus is arranged to perform measurements on a generic sample holder. The receptacle may be arranged to receive a sample holder, or the receptacle itself may be arranged to receive a sample.

According to a further aspect, a method for analysing a biological sample for diagnostics purposes is provided, in particular with an apparatus as described above, comprising the steps of:
providing a biological sample in a sample cartridge having least one sample reaction zone for containing said biological sample, in particular as defined above;
illuminating the at least one sample reaction zone of the sample cartridge;
performing measurements with a measurement section on the respective sample reaction zone of the sample cartridge, wherein a measurement section comprises at least one light detector for receiving light illuminated onto the respective sample reaction zone;
receiving and outputting data from the measurement sections.

The step of illuminating may comprise illuminating a reaction zone with light having different wavelengths.

As described above, the step of performing measurements preferably may comprise performing measurements on two respective sample reaction zones of a sample cartridge at the same time.

The respective reaction zones are then illuminated accordingly. The same light, in terms of wavelength may be used. Alternatively, the step of illuminating the at least two sample reaction zones comprises illuminating the first reaction zone with light having a first wavelength and illuminating the second reaction zone with light having a second wavelength, different from said first wavelength.

The present invention is further illustrated by the following Figures, which show a preferred embodiment of the apparatus according to the invention, and are not intended to limit the scope of the invention in any way, wherein:

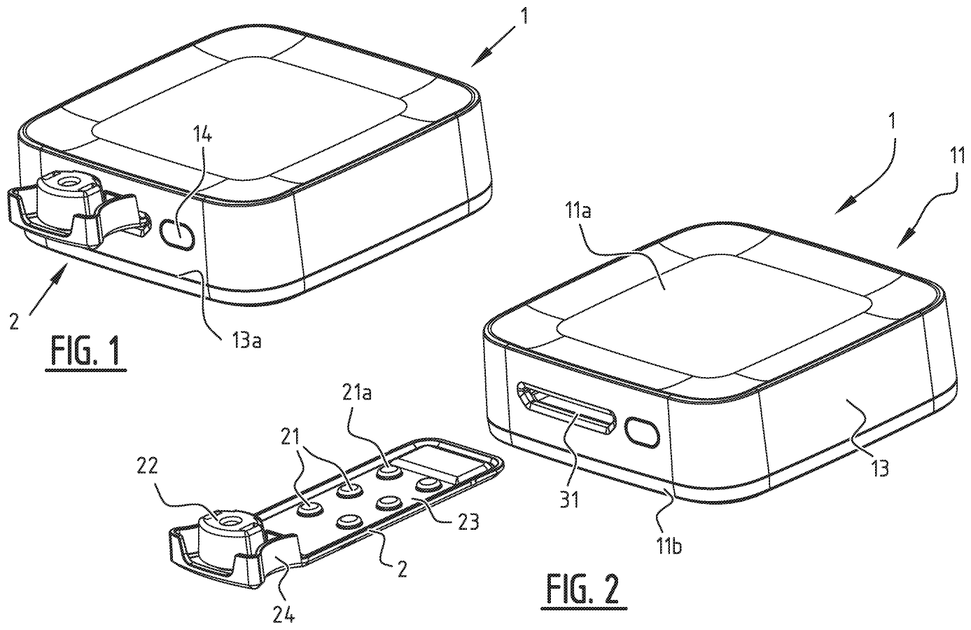

FIGS. 1 and 2 show a sample analysis apparatus with a sample cartridge inserted and with the cartridge removed, respectively;

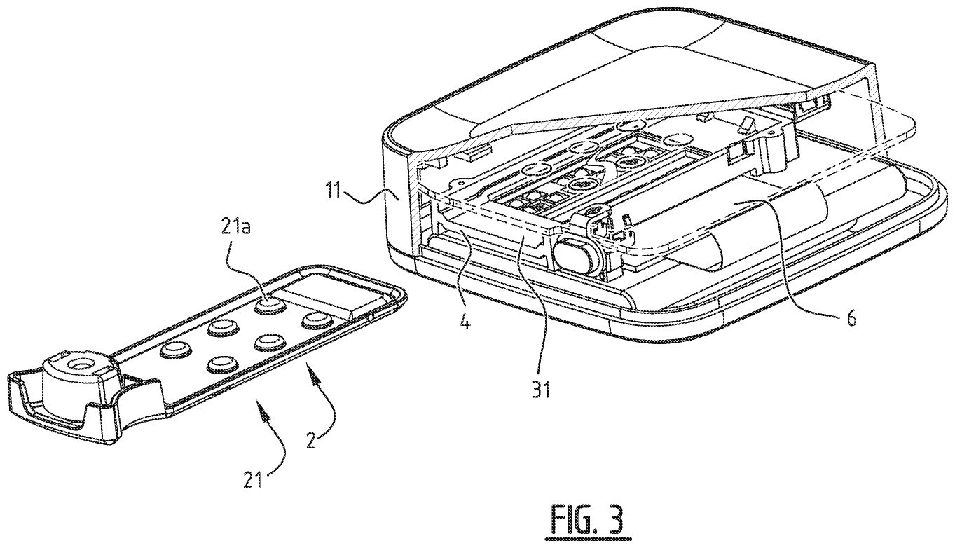

FIG. 3 shows internal components of the apparatus;

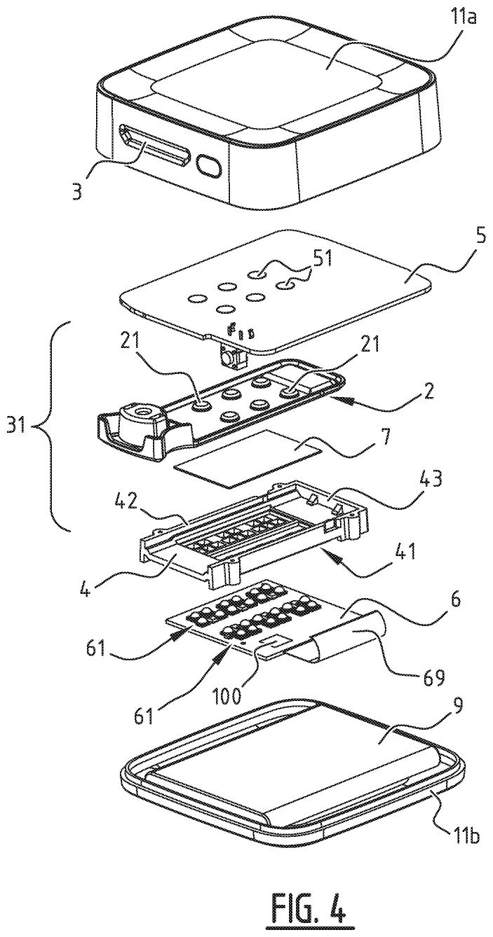

FIG. 4 shows the apparatus in exploded view;

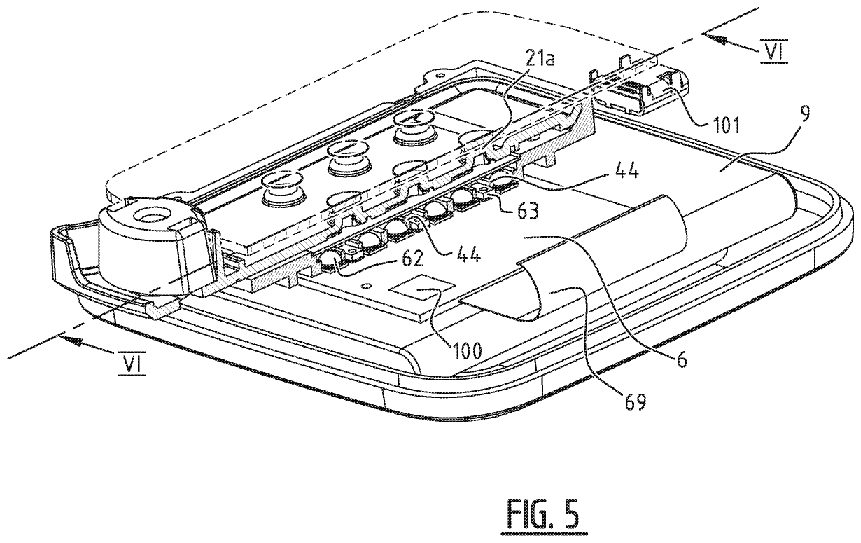

FIG. 5 shows the receptacle for the cartridge in perspective cross-sectional view;

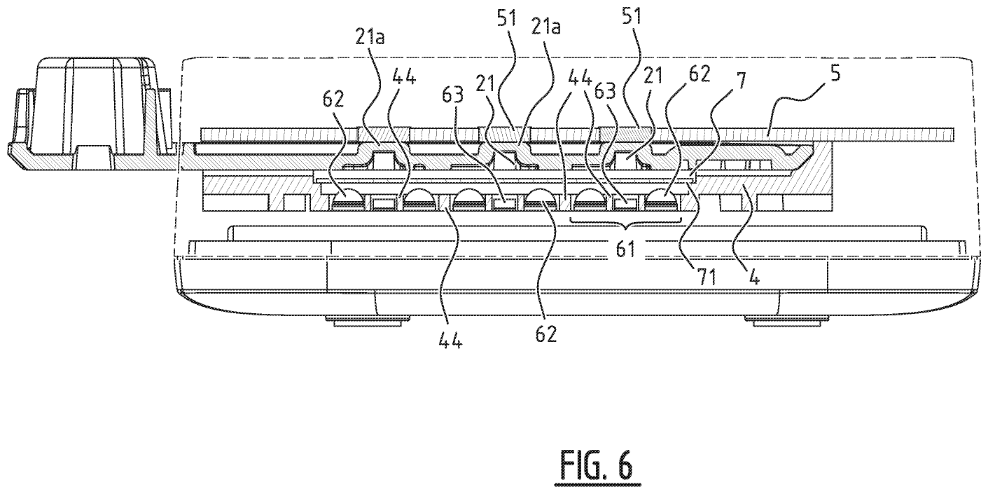

FIG. 6 shows a cross-sectional view along line VI as indicated in FIG. 5;

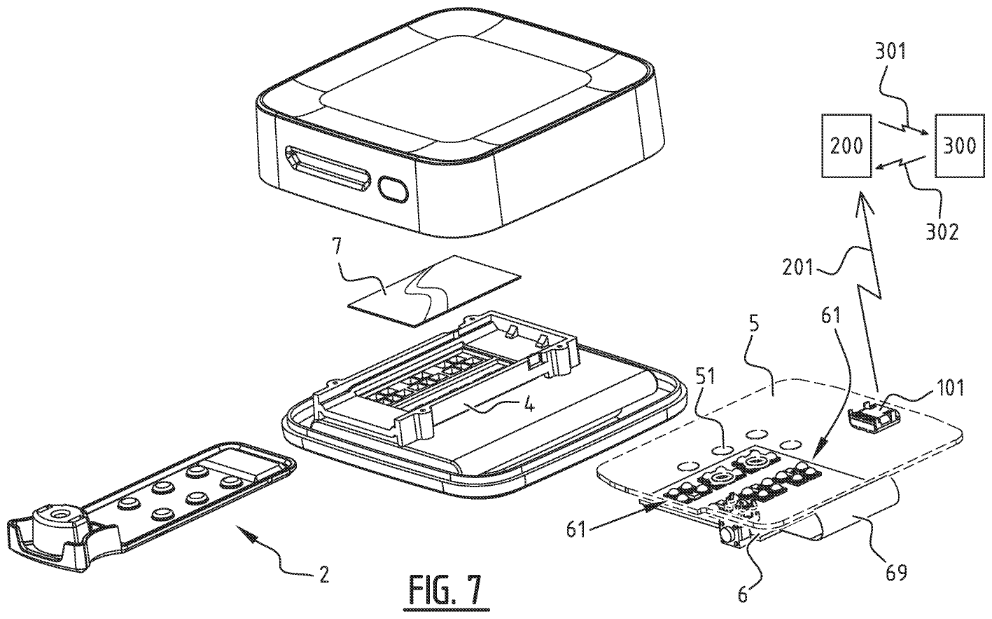

FIG. 7 shows parts of the apparatus forming the receptacle in perspective view;

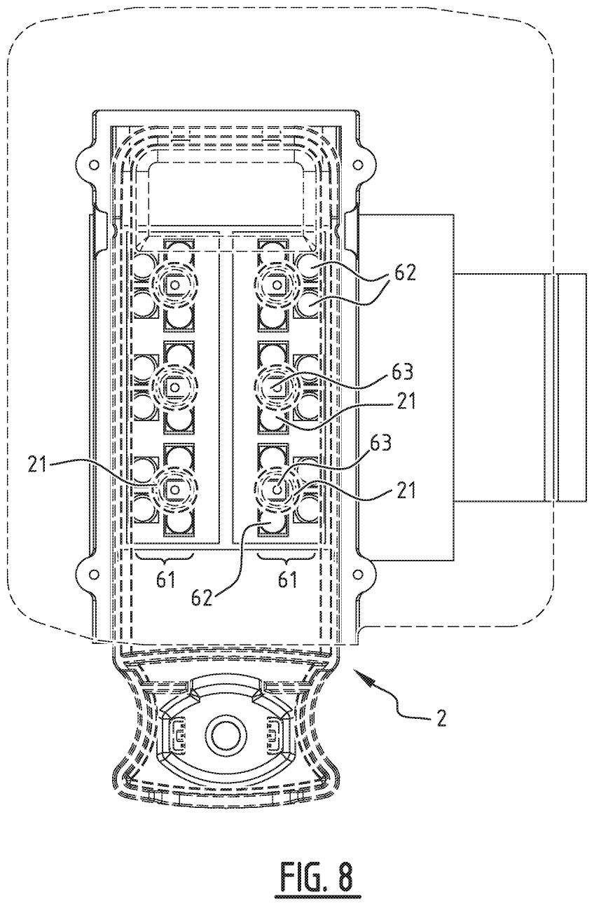

FIG. 8 shows the relative orientation of the cartridge and the measurement sections of the apparatus.

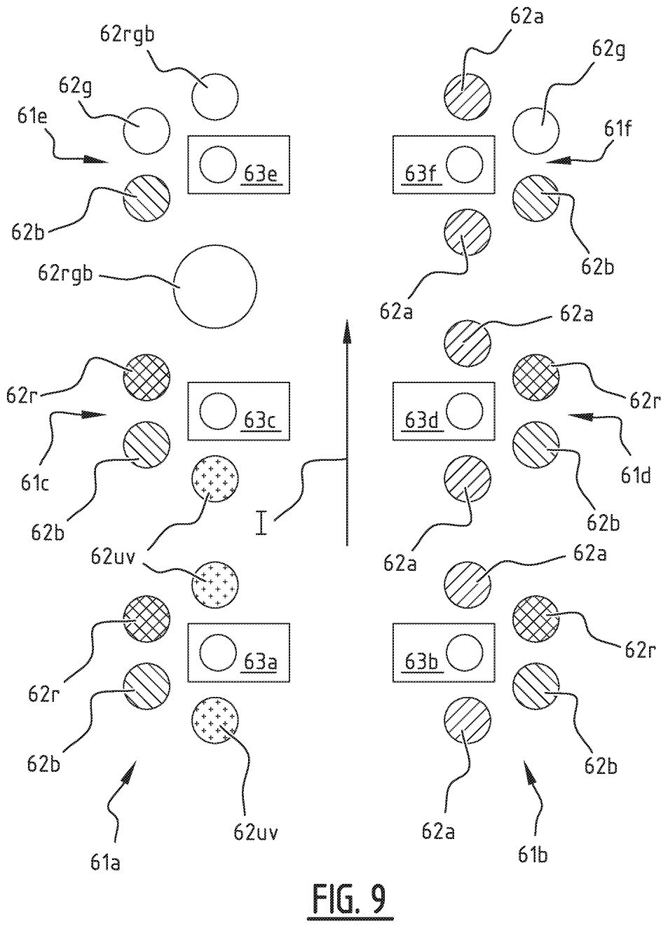

FIG. 9 shows an alternative configuration of a plurality of measurement sections.

With reference to in particular FIGS. 1-4, an apparatus 1 is shown which arranged to analyse a biological sample held in a cartridge 2. The cartridge 2 has a substantially planar configuration and comprises a base plate 23 onto which a plurality of reaction zones 21, in this example reaction chambers 21, are provided which are arranged to receive a biological sample via a closable inlet 22. The cartridge 2 comprises a plurality of reaction chambers 21, in this example six, which are preferably spatially separated from each other in both the X and Y-direction. The reactions chambers 21 are thus arranged in an array structure. In this example, the reaction chambers 21 are mutually coupled and contain the same biological sample. In the alternative, different samples may be contained in the respective reaction chambers 21.

The different reaction chambers 21 however contain different reagents to perform a plurality of different assays on the biological sample. A first chamber 21 may for instance be arranged to perform a PCR-test for detecting the presence of a first molecule or analyte, while a second reaction chamber 21 may be arranged to detect the presence of a second molecule of interest. The different chambers 21 may thus contain different reagents to amplify different molecules, thereby allowing the detection of different molecules of interest using a single cartridge 2. Different reaction chambers 21 may also be arranged for different tests such as a LAMP test, an antigen test, ELISA, a pH test or the like.

To be able to monitor the process of the different test and to perform measurements, as will be explained in greater detail below, each reaction chamber 21 is preferably provided with a transparent or at least translucent wall. In this example, the lower sides of the cartridge 2, and in particular at the locations of the reaction chambers 21 are transparent. The top wall 21a of the reaction chambers 21 are formed substantially planar (see also FIG. 6). This will improve the heat conduction between heating elements 51 (as will be explained in more detail below) and the sample contained in the reaction chamber 21.

As mentioned above, the cartridge 2 is generally plate shaped and is arranged to be inserted in a receptacle 31 of the apparatus 1, which is in this example slot shaped. To allow easy insertion of the cartridge 2 in the apparatus, a handle or gripper 24 near the inlet 22 is provided which is arranged to abut, in inserted position as shown in FIG. 1, a housing 11 of the apparatus 1. In this situation, the reaction chambers 21 extend inside the apparatus 1 and are located at the locations of different measurement and heating sections as will be explained below.

In this example, the housing 11 is formed by a top part 11a and a bottom part 11b (see FIG. 4), in between which different components are provided. In a front wall 13a of the top housing 11a, a slot 3 is provided which forms the entrance of the receptacle 31 arranged to receive the cartridge 2. Also provided on the front wall 13a is an activation button 14. Provided in the bottom part 11b is further a battery pack 9 for providing power to the components.

Generally, the apparatus 1 is arranged to perform measurements on the reaction chambers 21 of the sample cartridge 2 when the cartridge 2 is fully inserted into the receptacle 31. Opposite or underneath the reaction chambers 21 in the inserted position of the cartridge 2, the apparatus is provided with a plurality of measurement sections 61 (see FIG. 4). The positions of the measurement sections 61 correspond to the locations of the reaction chambers 21 of the cartridge 2, such that in inserted position, each reaction chamber 21 has associated therewith a measurement section 61. In this example, six measuring sections 61 are provided corresponding to the number of reaction chambers 21 on the cartridge 2.

In this example, the measurement sections 61 are positioned in the apparatus 1 to be located directly below (see for instance FIGS. 5 and 6) the respective reaction chambers 21 of the cartridge 2 when fully inserted. The measurement sections 61 can then efficiently perform measurements on the reaction chambers 21.

Each measurement section 61 is provided with a multi-spectral light sensor 63. The sensors 63 are in this example located directly below the (centre of the) reaction chambers 21. The sensors 63 are thus arranged to detect any light received from the reaction chambers 21, which may be provided with a transparent bottom as mentioned above to allow measurement of the contents of the reaction chamber 21 by the sensor 63.

To improve the detection accuracy, it is preferred if the reaction chambers 21 are illuminated. A light source is thereto provided. Although it may be possible to use one central or a shared light source in the apparatus, it is preferred when each measurement section 61 has a least one light source 62, in this example in the form of LEDs 62.

Each measurement section 61 comprises in this example a plurality of LEDs 62. At least two of the light sources 62 are arranged to emit light with mutually different wavelengths. In this example, all four LEDs 62 in a measurement section 61 emit light with different wavelengths. A LED 62 for illumining red, green, blue and white light or UV is provided in this non-limiting example. The LEDs 62 are positioned around the sensor 63 in a measurement section 61. The sample in a reaction chamber 21 located above a measurement section 61 can thus efficiently be illuminated by each of the LEDs 62 in a measurement section 61.

The measurement sections 61 are located on a frame member 6, in this example in the form of a PCB. Also a controller 100 is provided, in this example on the same PCB. The locations of the measurement sections 61 correspond to the locations of the reaction chambers 21 of the cartridge 2 such that in inserted position of the cartridge 2, the respective reaction zones 21 extend closely to the respective measurement section 61. In particular the light detectors or sensors 63 of the respective measurement sections 61 are positioned closely to, in this example directly under, the reaction chambers 21. This is best visible in FIG. 8. The light detectors 63 are arranged near the centre of a reaction chamber 21 of a cartridge 2 which is received in the receptacle, while the LEDs 62 are arranged to surround the light detectors 63.

To ensure that the cartridge 2 can only be correctly received in a predetermined manner, the receptacle 31 is arranged to closely receive the cartridge 2. With reference to in particular FIGS. 3 and 4, the receptacle 31 is formed by top frame member 5 and a lower frame 4. In assembled state as shown in FIG. 3, a slot-like receptacle 31 is formed between these members 4 and 5. The side and end walls of the receptacle are formed by side walls 42 and an end wall 43 of the frame member 4.

An intermediate section 41 of the frame member 4 between the entry of the receptacle 3 and the end wall 4 is provided with a plurality of through-holes which are arranged to receive the components of the measurement sections 61, i.e. the LEDs 62 and the light detectors 63, see FIG. 5. The LEDs 62 and optical sensors 63 extend in these through-holes. Thus, between the respective components 62, 63, light shielding walls 44 are provided, see also FIGS. 6 and 7.

On the upper frame member 5, heating elements 51 are provided. The locations of the heating elements 51 again correspond to the locations of the reaction chambers 21 of a cartridge 2 received in the receptacle 31. The heating elements 51 are arranged to heat the reaction chambers 21, in particular the planar upper walls 21a thereof. As best visible in FIG. 6, a heating element 51 is provided in close proximity of the upper wall 21a of a reaction chamber 21, in this example directly above it. The apparatus 1 is arranged to control each of the heating elements 51 individually, such that the temperature of each of the reaction chambers 21 can be controlled. Also the upper frame member 5 is formed as a PCB, such that the respective heating elements 51 can be controlled efficiently. The upper PCB 5 is coupled to the lower PCB 6 and therewith with the controller 100 via a flex-PCB 69 (see FIG. 4).

Also visible in for instance FIG. 4 is a transparent member 7 which forms the bottom of the receptacle in this example. The transparent member 7 extends between the cartridge 2 and the components of the measurement sections 61 to seal the electronics. The transparent member 7 is provided with a heat reflecting layer 71 (see FIG. 6). This prevents heat originating from the heating elements 51 reaching the measurement sections 61.

In inserted state of the cartridge 2, a heating element 51 is located at a first side, here above, a reaction chamber 21, while at a measurement section 61, in particular the light sensor 63 thereof, is located at the opposite side. The reaction chamber 21 thus extends between a heating element 51 and a measurement section 61.

In use, the controller 100 will control the respective measurement sections 61 to perform the required test for each reaction chamber 21. A measurement section 61 may thus perform different measurements, also on a single reaction chamber 21. Different measurement sections 61 may also use light with mutually different wavelengths to illuminate the respective reaction chambers 21. Also the light sensors 63 of the different measurement sections 61 may be controlled to record, or to detect the presence of, light with different wavelengths. The data is fed to the controller 100, which may feed the data to an output 101.

The light sensor, for instance in the form of a multi-spectral sensor, may also be arranged to detect light within a certain bandwidth of wavelengths and to provide the multi-spectral data to the controller 100 for output to the output 101.

The output 101 and/or the controller 100 may output the data directly to a remote server (schematically indicated with 300 in FIG. 7), but preferably transmits the data to an electronical device 200 of a user, for instance a smartphone or laptop. The data may be transferred wirelessly, for instance via Bluetooth, indicated with arrow 201. The device 200 may then transfer the data via a secure internet connection to the central server 300 where the data is analysed. Here, a diagnostic parameter being indicative for the results of the diagnostic test based on the sample in the sample cartridge may be calculated, which in turn may be provided back to the device 200 (arrow 302).

Where in the above example the measurement sections 61 each have a corresponding configuration, this is not required. In FIG. 9, a plurality of measurement sections 61a-f is shown, each provided with a respective multi-spectral sensor 63a-f. As follows from FIG. 9, the configurations of the measurement sections 61a-f differ.

For example, a part of the measurement sections 61b,d,f, in this example those on the right side of the apparatus 1 (as seen with respect to the insertion direction of the cartridge 2 indicated with the arrow I in FIG. 9), each comprise two LEDs 62a arranged to emit white light. These white lights 62a make these measurement sections 61b,d,f particularly suitable to detect any test lines of a lateral flow assay incorporated in a cartridge (not shown) or inserted directly in the receptacle 31. Each of the sensors 63b,d,f is then arranged to detect a test line of a lateral flow test.

Also blue LEDs 62b are provided. In this example, each of the measurement sections 61a-f comprises a blue LED 62b, emitting light with a wavelength of 450 nm. The blue LEDs 62b are arranged to excitation a fluorescence, for instance SYTO 9 or pico green.

A plurality of the measurement sections 61 a-d is further provided with a red LED 62r, emitting light with a wavelength of 620 nm in this example. The red LEDs 62r are for instance used to excite fluorescence such as Cy 5. A plurality of the measurement sections 61e,f further comprises green LEDs 62g, emitting light with a wavelength of 525 nm in this example, which may also be used to excite fluorescence (Cy 3).

Also provided are multispectral light sources 62rgb. In this example, the light sources 62rgb comprise a RGBW LED, which is thus also arranged to emit white light, next to red, green and blue light. These light sources can be controlled to emit a light with any desired colour, including white light. These light sources 62rgb are again beneficial in performing measurements on a lateral flow test. One of the multispectral light sources is relatively large and is shared by measurement sections 61c and 63e and is located therebetween. In other words, the light source 62rgb can be controlled for a measuring operation for both measurement sections 61c and 61e.

The present invention is not limited to the embodiment shown, but extends also to other embodiments falling within the scope of the appended claims.

1-45. (canceled)

46. An apparatus for analysing a biological sample for diagnostics purposes, the apparatus comprising:
a sample cartridge receptacle for receiving a sample cartridge for holding the biological sample, wherein the sample cartridge comprises at least two sample reaction zones for containing said biological sample;
at least one light source associated with the sample cartridge receptacle for illuminating the at least two sample reaction zones of a sample cartridge when received in the sample cartridge receptacle;
at least two measurement sections associated with the sample cartridge receptacle, wherein each of the at least two measurement sections is arranged to perform measurements on a respective sample reaction zone of the at least two sample reaction zones of a sample cartridge when received in the sample cartridge receptacle, wherein each measurement section comprises at least one light detector for receiving light illuminated onto the respective sample reaction zone by the at least one light source;
a processing unit arranged for controlling the at least one light source and for receiving data from the measurement sections.

47. The apparatus according to claim 46, wherein each measurement section comprises a respective light source for illuminating the respective reaction zone of a sample cartridge when received in the sample cartridge receptacle, and/or wherein at least two of the light sources are arranged to emit light with different wavelengths.

48. The apparatus according to claim 47, wherein at least two measurement sections comprise at least two light sources arranged to emit light with different wavelengths, wherein optionally the processing unit is arranged to individually control the at least two light sources for illuminating the at least two sample reaction zones with light having different wavelengths and to receive data from the respective measurement sections obtained from illumination of the respective sample reaction zones illuminated with light having mutually different wavelengths.

49. The apparatus according to claim 46, wherein the processing unit is arranged to control at least two measurement sections for simultaneously taking measurements on two sample reactions zones of a sample cartridge when received in the sample cartridge receptacle.

50. The apparatus according to claim 46, wherein the light detector comprises a multi-spectral sensor, wherein optionally the processing unit is arranged to receive multi-spectral data from the sensor.

51. The apparatus according to claim 46, wherein at least two measurement sections have a different configuration.

52. The apparatus according to claim 46, further comprises a Printed Circuit Board (PCB), wherein the light source and the light detector are arranged on the PCB.

53. The apparatus according to claim 52, wherein at least two measurement sections have a different configuration, and wherein the PCB comprises a plurality of light detectors and light sources for defining a plurality of measurement sections, optionally further comprising a light shielding mechanism arranged between at least two measurement sections.

54. The apparatus according to claim 46, further comprising a light shielding mechanism arranged between a light source and a light sensor.

55. The apparatus according to claim 46, wherein the sample cartridge receptacle is at least partly defined by a transparent member arranged between at least the light detector in a measurement section and a sample cartridge when received in the sample cartridge receptacle, wherein optionally the transparent member comprises a heat reflecting layer.

56. The apparatus according to claim 46, further comprising at least one heating element arranged for heating a respective sample reaction zone of a sample cartridge when received in the sample cartridge receptacle, wherein the processing unit is arranged for controlling the at least one heating element, wherein optionally:
at least two measurement sections have associated therewith a respective heating element arranged for heating the respective sample reaction zones of a sample cartridge when received in the sample cartridge receptacle, wherein the processing unit is arranged for individually controlling the at least two heating elements; and/or
the apparatus further comprises a heat sink associated with the light detector; and/or
the measurement section is arranged at first side of the sample cartridge receptacle and wherein the heating element is arranged at an opposite side of the sample cartridge receptacle for receiving a sample cartridge therebetween.

57. The apparatus according to claim 46, wherein the sample cartridge receptacle is arranged for receiving a sample cartridge comprising a plurality of sample reaction zones for containing said biological sample, wherein the reaction zones are arranged in a two-dimensional array configuration, wherein the reaction zones are separated in a first direction and a second direction orthogonal to the first direction optionally comprising a plurality of measurement sections, wherein the measurement sections are arranged in a corresponding two-dimensional array configuration.

58. The apparatus according to claim 46, further comprising a memory, wherein the memory contains instructions for controlling the light source, the detection section and optionally the heating element, for performing predetermined measurements on a reaction zone of a received sample cartridge.

59. The apparatus according to claim 46, wherein the apparatus is arranged to perform a plurality of different measurements including at least two of a PCR test, LAMP test, antigen test, ELISA, pH test, a (neutralizing) antibody test and any other reaction based test that results in a change in the optical properties of a sample.

60. The apparatus according to claim 46, further comprises an output module for outputting the received data from the measurement sections, wherein optionally the output module is a wireless module for wirelessly transmitting the data.

61. The apparatus according to claim 46, wherein the apparatus is a handheld device and/or the apparatus further comprising a battery.

62. A system for analysing a biological sample for diagnostics purposes comprising an apparatus according to claim 46 and a central server, wherein the apparatus is arranged to provide the received data from the measurement sections to the central server, wherein the central server is arranged to analyse the received data and to provide a diagnostic parameter being indicative for the results of the diagnostic test based on the sample in the sample cartridge, wherein optionally the central server comprises an output for outputting the diagnostic parameter to a user.

63. The system according to claim 62, wherein the central server is arranged to receive the data via an electronical device from a user, such as a mobile phone, a tablet or a laptop, coupled to the apparatus.

64. A sample cartridge for use in the apparatus according to claim 46, wherein the sample cartridge comprises at least two sample reaction zones for containing said biological sample, wherein optionally the reaction zones are arranged in a two-dimensional array configuration, wherein the reaction zones are separated in a first direction and a second direction orthogonal to the first direction.

65. The sample cartridge according to claim 64, wherein at least two reaction zones are arranged for two different assays or tests and/or wherein a reaction zone comprises a heat conducting section for cooperating with a heating element and/or wherein a reaction zone comprises a substantially planar wall of heat conducting material.

66. The sample cartridge according to claim 64, wherein at least one of the reaction zones is arranged for a PCR test, LAMP test, antigen test, ELISA, pH test, a (neutralizing) antibody test and any other reaction based test that results in a change in the optical properties of a sample.

67. A method for analysing a biological sample for diagnostics purposes with an apparatus according to claim 46, comprising the steps of:
providing a biological sample in a sample cartridge having least one sample reaction zone for containing said biological sample;
illuminating the at least one sample reaction zone of the sample cartridge;
performing measurements with a measurement section on the respective sample reaction zone of the sample cartridge, wherein a measurement section comprises at least one light detector for receiving light illuminated onto the respective sample reaction zone; and
receiving and outputting data from the measurement sections.

68. The method according to claim 67, wherein the sample cartridge comprises at least two reaction zones, wherein the step of performing measurements comprises performing measurements on two respective sample reaction zones at the same time with two respective measurement sections, wherein optionally the step of illuminating comprises illuminating a reaction zone with light having different wavelengths.

69. The method according to claim 67, wherein the sample cartridge comprises at least two reaction zones, wherein the step of illuminating comprises illuminating the at least two sample reaction zones.
